(19) United States
(12) Patent Application Publication
(10) Pub. No.: US 2018/0078114 A1
(43) Pub. Date: Mar. 22, 2018

(54) ENDOSCOPE AND OPTICAL TRANSMISSION MODULE

(71) Applicant: OLYMPUS CORPORATION, Tokyo (JP)

(72) Inventor: Keiichi KOBAYASHI, Kamiina-gun (JP)

(73) Assignee: OLYMPUS CORPORATION, Tokyo (JP)

(21) Appl. No.: 15/811,952

(22) Filed: Nov. 14, 2017

Related U.S. Application Data
(63) Continuation of application No. PCT/JP2015/065232, filed on May 27, 2015.

(51) Int. Cl. A61B 1/00; A61B 1/05; H04N 5/225

(52) U.S. Cl. CPC A61B 1/00013; H04N 2005/2255; H04N 5/2256; A61B 1/05

(57) ABSTRACT

An optical transmission module includes: an optical fiber; a light emitting element; a holding member having a through-hole into which the optical fiber is inserted; a wiring board having a first principal surface onto which the holding member is joined, and a second principal surface on which a bond electrode and an external electrode of the light emitting element are disposed and bonded together; and a sealing resin for sealing a bond portion between the external electrode and the bond electrode. The optical transmission module further includes an intermediate member having an upper surface to be brought into contact with a distal end face of the optical fiber, and a lower surface to be brought into contact with a light emitting portion of the light emitting element.

### CROSS REFERENCE TO RELATED APPLICATION

This application is a continuation application of PCT/JP2015/065232 filed on May 27, 2015, the entire contents of which are incorporated herein by this reference.

### BACKGROUND OF INVENTION

### 1. Field of the Invention

The present invention relates to an optical transmission module including: an optical fiber configured to transmit an optical signal; an optical element; a holding member having a through-hole into which the optical fiber is inserted; and a wiring board including a hole portion functioning as an optical path for the optical signal, and having a first principal surface to be joined with the holding member, and a second principal surface on which the optical element is mounted. The present invention also relates to an endoscope including the optical transmission module at a distal end portion of an insertion portion.

### 2. Description of the Related Art

An endoscope includes an image pickup device, such as a CCD, at a distal end portion of an elongated flexible insertion portion. In recent years, use of an image pickup device having a larger number of pixels for an endoscope has been studied. When an image pickup device having a large number of pixels is used, the number of signals to be transmitted from the image pickup device to a signal processing device (processor) increases. Accordingly, optical signal transmission through an elongated optical fiber using optical signals is preferably employed instead of electric signal transmission through a metal wire using electric signals. In the optical signal transmission, an E/O optical transmission module (electricity-to-light converter) for converting an electric signal into an optical signal, and an O/E optical transmission module (light-to-electricity converter) for converting an optical signal into an electric signal are used.

Accurate positioning and fixation are important for the optical transmission modules to achieve effective optical coupling between an optical element and an optical fiber through which optical signals are transmitted. To accurately and easily perform positioning of the optical element and the optical fiber, a holding member (ferrule) including a through-hole formed in a wiring board on which the optical element is mounted is used for the optical transmission module. The optical fiber is inserted into the through-hole of the holding member, thereby facilitating the positioning of the optical element and the optical fiber in the horizontal direction. To accurately perform positioning, the diameter of the through-hole is set to be slightly larger than the outer diameter of the optical fiber.

Japanese Patent Application Laid-Open Publication No. 2014-10329 discloses that a hole portion of a wiring board on which an optical element is mounted is tapered, and a distal end face of an optical fiber is brought into contact with the hole portion so that the distance between the optical element and the end face of the optical fiber, i.e., the vertical direction, can be positioned accurately and easily.

### SUMMARY OF THE INVENTION

An endoscope according to an embodiment includes: an insertion portion including an optical transmission module at a distal end portion in which an image pickup device is disposed; and an operation portion extending toward a proximal end portion side of the insertion portion. The optical transmission module includes: an optical fiber including a core portion through which an optical signal is transmitted, and a clad portion covering an outer circumferential surface of the core portion, the optical fiber being inserted through the insertion portion; an optical element having a front surface on which an optical element portion and an external electrode are disposed, the optical signal being output from the optical element portion, or being incident on the optical element portion; a holding member having a through-hole into which the optical fiber is inserted; a wiring board including a hole portion functioning as an optical path for the optical signal, the holding member being joined to a first principal surface, the external electrode of the optical element and a bond electrode being disposed on a second principal surface and bonded together; and a sealing resin for sealing a bond portion between the external electrode and the bond electrode. The endoscope further includes an intermediate member having an upper surface to be brought into contact with a distal end face of the optical fiber, and a lower surface to be brought into contact with the optical element portion of the optical element, and having a same configuration as a configuration of the optical fiber. An upper portion of the intermediate member is inserted into the through-hole of the holding member, a perimeter of the upper portion of the intermediate member being tapered.

An optical transmission module according to another embodiment includes: an optical fiber configured to transmit an optical signal; an optical element having a front surface on which an optical element portion and an external electrode are disposed, the optical signal being output from the optical element portion, or being incident on the optical element portion; a holding member having a through-hole into which the optical fiber is inserted; a wiring board including a hole portion functioning as an optical path for the optical signal, the holding member being joined to a first principal surface, the external electrode of the optical element and a bond electrode being disposed on a second principal surface and bonded together; and a sealing resin for sealing a bond portion between the external electrode and the bond electrode. The optical transmission module further includes an intermediate member having an upper surface to be brought into contact with a distal end face of the optical fiber, and a lower surface to be brought into contact with the optical element portion of the optical element.

### BRIEF DESCRIPTION OF THE DRAWINGS

FIG. 1 is a perspective view illustrating an endoscope according to a first embodiment;

FIG. 2 is a sectional view illustrating an optical transmission module according to the first embodiment;

FIG. 3 is a sectional view illustrating a production method for the optical transmission module according to the first embodiment;

FIG. 4 is a sectional view illustrating the production method for the optical transmission module according to the first embodiment;

FIG. 5A is a perspective view illustrating an intermediate member of the optical transmission module according to the first embodiment;

FIG. 5B is a perspective view illustrating an intermediate member of the optical transmission module according to a modification of the first embodiment;

FIG. 5C is a perspective view illustrating an intermediate member of the optical transmission module according to another modification of the first embodiment;

FIG. 5D is a perspective view illustrating an intermediate member of the optical transmission module according to a still another modification of the first embodiment;

FIG. 6 is a sectional view illustrating an optical transmission module according to a second embodiment;

FIG. 7 is a partial sectional view illustrating the optical transmission module according to the second embodiment;

FIG. 8A is a partial sectional view illustrating an optical transmission module according to a modification of the second embodiment;

FIG. 8B is a partial sectional view illustrating an optical transmission module according to another modification of the second embodiment;

FIG. 9 is a sectional view illustrating an optical transmission module according to a third embodiment;

FIG. 10 is a sectional view illustrating a production method for the optical transmission module according to the third embodiment;

FIG. 11 is a sectional view illustrating a production method for the optical transmission module according to the third embodiment;

FIG. 12 is a sectional view illustrating an optical transmission module according to a fourth embodiment; and

FIG. 13 is a sectional view illustrating an optical transmission module according to a fifth embodiment.

### DETAILED DESCRIPTION OF THE PREFERRED EMBODIMENTS

### First Embodiment

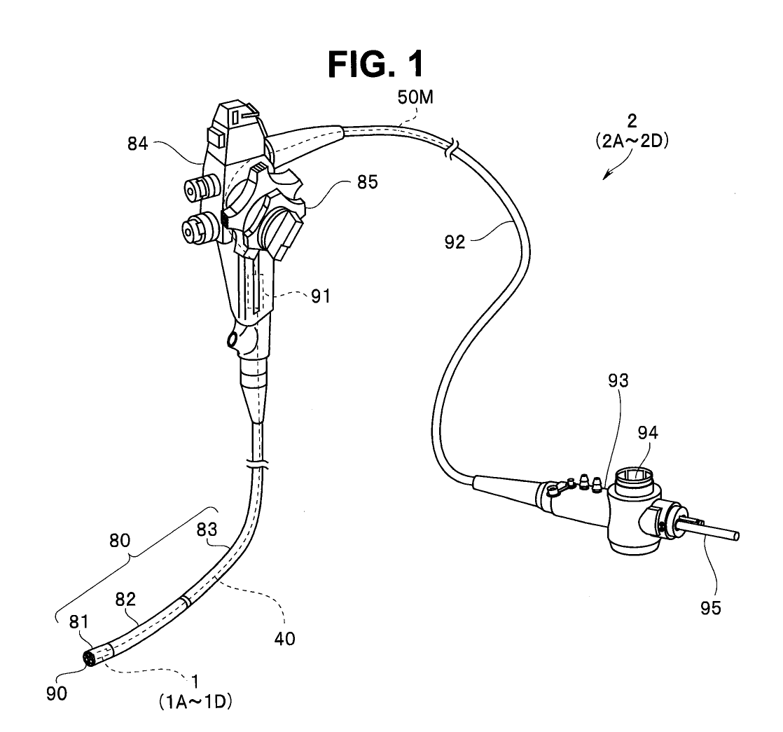

As illustrated in FIG. 1, an endoscope 2 according to this embodiment includes an insertion portion 80, an operation portion 84 disposed at a proximal end portion side of the insertion portion 80, a universal cord 92 extending from the operation portion 84, and a connector 93 disposed at a proximal end portion side of the universal cord 92.

The insertion portion 80 has a configuration in which a rigid distal end portion 81, a bending portion 82 for changing the direction of the distal end portion 81, and an elongated flexible portion 83 are sequentially connected.

The distal end portion 81 is provided with an image pickup optical unit 90L, an image pickup device 90, and an optical transmission module 1 which is an E/O module for converting an image pickup signal (electric signal) from the image pickup device 90 into an optical signal. The image pickup device 90 is a CMOS (complementary metal oxide semiconductor) image sensor, a CCD (charge coupled device), or the like.

The operation portion 84 is provided with an angle knob 85 for operating the bending portion 82, and an O/E module 91 which is an optical transmission module for converting an optical signal into an electric signal. The connector 93 includes an electric connector portion 94 to be connected to a processor (not illustrated), and a light guide connection portion 95 to be connected to a light source. The light guide connection portion 95 is connected to an optical fiber bundle for guiding illuminating light to the rigid distal end portion 81. Note that in the connector 93, the electric connector portion 94 and the light guide connection portion 95 may be integrally formed.

In the endoscope 2, the image pickup signal is converted into an optical signal by the optical transmission module 1, which is an E/O module disposed at the distal end portion 81, or the like, and is transmitted to the operation portion 84 through a thin optical fiber 40 that is inserted through the insertion portion 80. Further, the optical signal is converted into an electric signal again by the O/E module 91 disposed in the operation portion 84, and is transmitted to the electric connector portion 94 through a metal wire 50M that is inserted through the universal cord 92. Specifically, a signal is transmitted through the optical fiber 40 in the thin insertion portion 80, and the signal is transmitted through the metal wire 50M which is thicker than the optical fiber 40 in the universal cord 92 that is less limited in an outer diameter without being inserted into a body.

Note that, when the O/E module 91 is disposed in the vicinity of the electric connector portion 94, the optical fiber 40 may be inserted through the universal cord 92 to the vicinity of the electric connector portion 94. When the O/E module 91 is disposed in the processor, the optical fiber 40 may be inserted to the connector 93.

The endoscope 2 performs optical signal transmission through the thin optical fiber 40 using optical signals, instead of performing electric signal transmission, and thus the insertion portion 80 is thin and has minimal invasiveness.

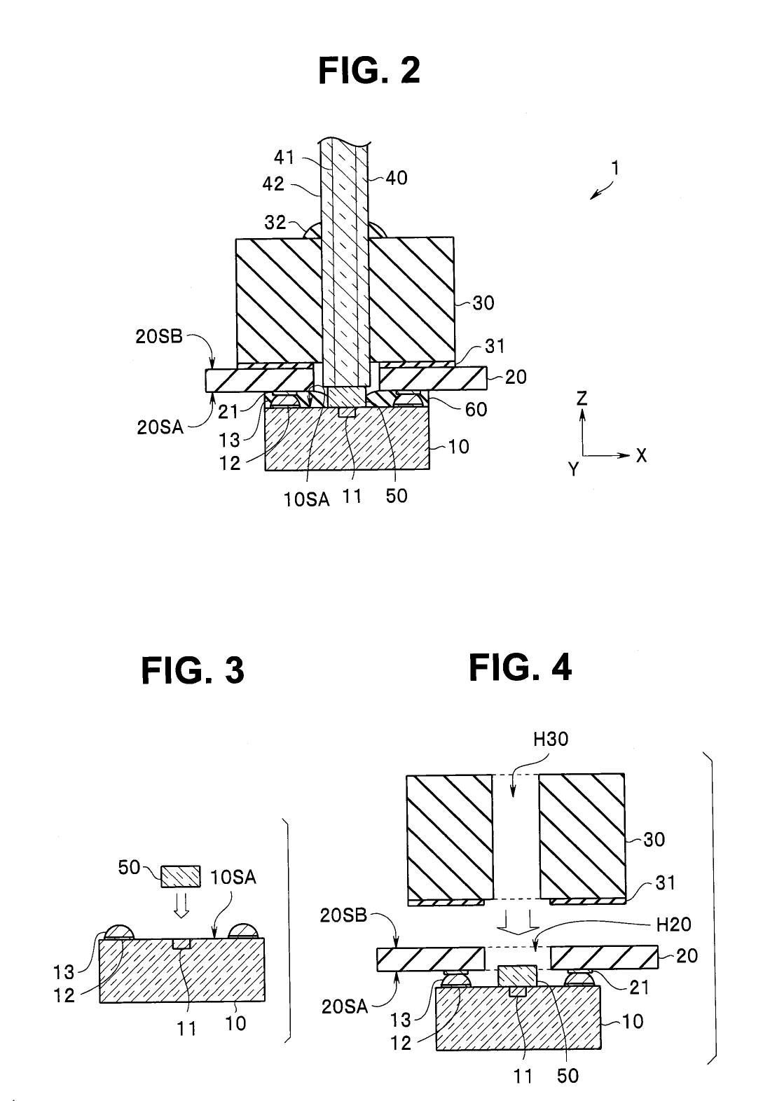

As illustrated in FIG. 2, the optical transmission module 1 according to this embodiment includes an optical element 10, which is a light emitting element, a wiring board 20, a holding member (also referred to as a ferrule) 30, the optical fiber 40 that is inserted through the insertion portion 80, and an intermediate member 50 that is made of glass. In the optical transmission module 1, the optical element 10, the wiring board 20, and the holding member 30 are arranged in a row in a thickness direction (Z-direction) of the optical element 10.

Note that, in the following description, the drawings based on the embodiments are schematic, and it should be noted that the relation between a thickness and a width of each part, a ratio between thicknesses of respective parts, a relative angle therebetween, and the like are different from actual ones. The relation or ratio between the dimensions of respective parts may be different in the drawings. A direction in which the value of the Z-axis increases is referred to as “upper”.

The optical element 10 is a surface-emitting laser chip formed on a light emitting surface 10SA, a front surface of which corresponds to a light emitting portion 11 which is an optical element portion that outputs light of an optical signal. For example, the light emitting surface 10SA of the micro optical element 10, which has dimensions in plan view of 250 μm×300 μm, is provided with the light emitting portion 11, which has a diameter of 20 μm, and external electrodes 12 which supply a drive signal to the light emitting portion 11.

On the other hand, for example, the optical fiber 40 includes a core portion 41 that transmits light and has a diameter of 50 μm, and a clad portion 42 that covers the outer circumferential surface of the core portion 41 and has a diameter of 125 μm. The core portion 41 is made of glass which has a refractive index slightly smaller than that of the clad portion 42 by, for example, about 0.2% to 0.3%.

The holding member 30 which is joined onto the optical element 10 and has a substantially rectangular parallelepiped shape has a through-hole H30 into which the distal end portion of the optical fiber 40 is inserted. The optical fiber 40 is inserted and fitted into the through-hole H30, thereby positioning the optical fiber 40 and the light emitting portion 11 of the optical element 10. The inside shape of the through-hole H30 may be a columnar shape, or a prism shape, such as a quadrangular prism shape or a hexagonal prism shape, as long as the wall surface of the through-hole H30 can hold the optical fiber 40. A material for the holding member 30 is ceramic, silicon, glass, a metallic member such as SUS, or the like. Note that the holding member 30 may have a substantially columnar shape, a substantially conical shape, or the like.

As described above, the holding member 30 is provided with the columnar through-hole H30, a diameter (inner diameter) R30 of which is substantially the same as an outer diameter R40 of the optical fiber 40 to be inserted. The phrase “substantially the same” used herein means that the diameter of the optical fiber 40 and the diameter of the through-hole H30 are set to be substantially “the same” size so that the outer circumferential surface of the optical fiber 40 and the wall surface of the through-hole H30 are brought into contact with each other and fitted each other.

For example, the diameter R30 of the through-hole H30 is formed in a size that is larger by 1 μm to 5 μm than the outer diameter R40 of the optical fiber 40.

A hole portion H2O functioning as an optical path is present in the flat wiring board 20 including a first principal surface 20SA and a second principal surface 20SB. Bond electrodes 21, which are disposed on the first principal surface 20SA of the wiring board 20, and the external electrodes 12 of the optical element 10 are bonded together via bumps 13. In other words, the optical element 10 is flip-chip mounted on the wiring board 20 in a state where the light emitting portion 11 is disposed in a position opposed to the hole portion H20 of the wiring board 20. Accordingly, a gap corresponding to the height of each bump 13 is formed between the light emitting portion 11 of the optical element 10 and the first principal surface 20SA of the wiring board 20. For example, stud gold bumps 13 may be respectively ultrasound-bonded to the bond electrodes 21 of the wiring board 20.

A bond portion between the external electrodes 12 of the optical element 10 and the bond electrodes 21 of the wiring board 20 is sealed by a sealing resin 60, such as epoxy resin or silicone resin, which has excellent moisture resistance and insulation properties.

As a base of the wiring board 20, an FPC substrate, a ceramic substrate, a glass epoxy substrate, a glass substrate, a silicon substrate, or the like is used.

Note that, after printing solder paste or the like as bumps and disposing the optical element 10 in a predetermined position, solder may be melted by a reflow or the like to mount the optical element 10 on the wiring board 20. Note that the wiring board 20 may include a processing circuit or the like for converting an electric signal transmitted from the image pickup device 90 into a drive signal for the optical element 10.

The holding member 30 is joined to the second principal surface 20SB of the wiring board 20 by an adhesive layer 31 in a state where the through-hole H30 is disposed in a position opposed to the hole portion H20.

The upper surface of the intermediate member 50 is brought into contact with the distal end face of the optical fiber 40, and the lower surface of the intermediate member 50 is brought into contact with the light emitting portion 11 of the optical element 10. The intermediate member 50 that constitutes an optical path for an optical signal is made of, for example, glass, which transmits light of the optical signal.

As illustrated in FIG. 3, in a production method for the optical transmission module 1, the intermediate member 50 is first joined to the light emitting portion 11 of the light emitting surface 10SA of the optical element 10 with a transparent adhesive (not illustrated).

Note that in the optical transmission module 1 including a light emitting element as the optical element 10, the light emitting portion 11 is preferably covered completely by the lower surface of the intermediate member 50 so as to effectively guide the light generated by the optical element 10 to the optical fiber 40.

The intermediate member 50 may be formed of a transparent resin, such as silicone resin, epoxy resin, or acrylic resin, as long as the material can favorably transmit the wavelength of the optical signal. When the wavelength of the optical signal corresponds to an infrared wavelength, the intermediate member does not transmit visible light, but instead may be formed of a material that transmits infrared light, such as silicon.

Next, as illustrated in FIG. 4, the wiring board 20 and the optical element 10 are first bonded together. Specifically, the bond electrodes 21 of the first principal surface 20SA of the wiring board 20 and the external electrodes 12 of the optical element 10 are bonded together via the gold bumps 13.

After that, a liquid sealing resin is injected into the bond portion between each bond electrode 21 and each external electrode 12 and is cured. When the sealing resin is spread to the light emitting portion of the optical element, the amount of light guided to the optical fiber may decrease and the efficiency of bonding between the optical fiber and the optical element may deteriorate. However, in the optical transmission module 1, the light emitting portion 11 is covered with the intermediate member 50, so there is no possibility that the uncured sealing resin may spread to the light emitting portion 11.

A resin that does not transmit light of an optical signal and has a light blocking function may be used as the sealing resin.

After that, the holding member 30 is joined to the back surface 20SA of the substrate 20 on which the optical element 10 is mounted. Further, the distal end face of optical fiber 40 is inserted and fitted into the through-hole H30 of the holding member 30 until the distal end face reaches a position where the distal end face is brought into contact with the upper surface of the intermediate member 50. The distance between the distal end face of the optical fiber 40 and the light emitting portion 11 of the optical element 10 is accurately positioned by the height of the holding member 30, and is fixed using an adhesive 32.

Accordingly, the optical transmission module 1 has a high efficiency in bonding between the optical fiber 40 and the optical element 10 and can be easily produced.

### <Modification of First Embodiment>

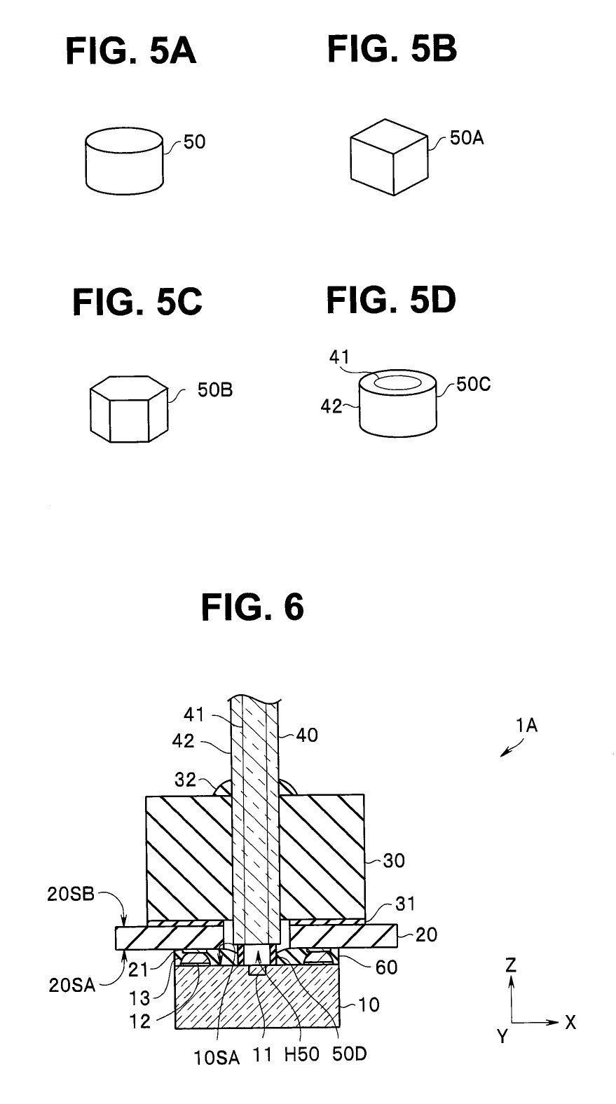

Note that the shape of the intermediate member is not limited to the columnar intermediate member 50 illustrated FIG. 5A, as long as the lower surface of the intermediate member covers the light emitting portion 11. For example, an intermediate member 50A which has a quadrangular prism shape as illustrated in FIG. 5B, or an intermediate member 50 which has a polygonal columnar shape as illustrated in FIG. 5C may be used.

It is particularly preferable to use the intermediate member 50C having the same configuration as that of the optical fiber 40, as illustrated in FIG. 5D. Specifically, like the optical fiber 40, the intermediate member 50C which includes the clad portion 42 that covers the outer circumferential surface of the core portion 41 can be easily prepared by cutting the optical fiber 40 into a predetermined length, specifically, a length corresponding to the distance between the distal end face of the optical fiber 40 and the light emitting portion 11 of the optical element 10. Further, the intermediate member 50C having the same configuration as that of the optical fiber 40 has a high light transmission efficiency.

Note that, in order to effectively guide the light generated by the optical element 10 to the optical fiber 40, the light emitting portion 11 is preferably covered completely by the lower surface of the core portion 41 of the intermediate member 50C.

### Second Embodiment

An optical transmission module 1A according to a second embodiment and an endoscope 2A including the optical transmission module 1A will be described. The optical transmission module 1A and the endoscope 2A are similar to the optical transmission module 1 and the endoscope 2, respectively, and have the same functions as those of the optical transmission module 1 and the endoscope 2. Accordingly, the same components are denoted by the same reference numerals, and descriptions thereof are omitted.

As illustrated in FIG. 6, in the optical transmission module 1A, an intermediate member 50D has a tubular shape, in other words, a hollow cylinder shape, and has a through-hole H50 which is a space functioning as an optical path and formed at the center of the intermediate member. The intermediate member 50D is made of metal, ceramic, resin, or the like.

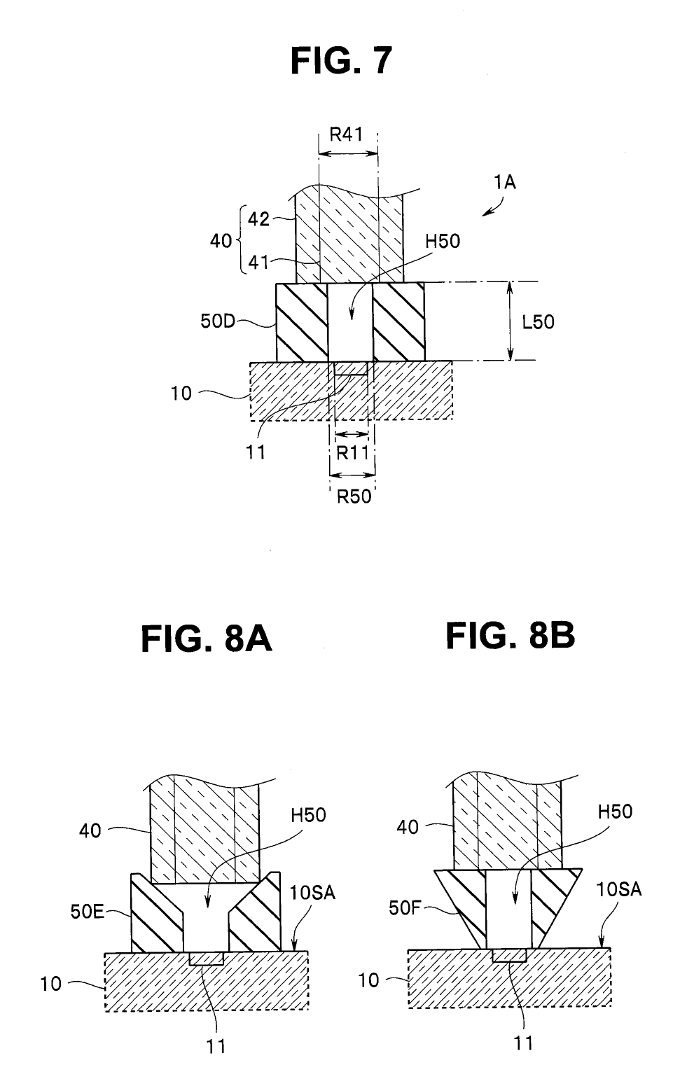

As illustrated in FIG. 7, in the optical transmission module 1A including a light emitting element as the optical element 10, a diameter R50 of the through-hole H50 of the intermediate member 50D is preferably larger than an outer diameter R11 of the light emitting portion 11 and smaller than an outer diameter R41 of the core portion 41 of the optical fiber 40. Note that, in the optical transmission module including a light receiving element as the optical element 10, the diameter R50 of the through-hole H50 of the intermediate member 50D is preferably smaller than the outer diameter R11 of the light receiving portion and larger than the outer diameter R41 of the core portion 41 of the optical fiber 40. Note that a length L50 of the intermediate member 50D is smaller than the distance between the light receiving surface 10SA of the optical element 10 and the lower surface of the holding member 30.

The optical transmission module 1A can be produced at a cost lower than that of the optical transmission module 1.

Note that the inside of the through-hole H50 may be filled with a transparent resin such as silicone resin, epoxy resin, or acrylic resin. In this case, the intermediate member 50D is similar to the intermediate member 50.

### <Modification of Second Embodiment>

In an intermediate member 50E illustrated in FIG. 8A, an opening of an upper surface of the through-hole H50 is tapered. A distal end face of the optical fiber 40 is brought into contact with the tapered surface. The intermediate member 50E allows the optical fiber 40 to be easily disposed perpendicularly to the light emitting surface 10SA of the optical element 10.

An outer circumferential surface of an intermediate member 50F illustrated in FIG. 8B has a tapered shape toward the lower surface thereof. Even when the external electrodes 12 is disposed near the light emitting portion 11 of the optical element 10, the intermediate member 50F can be easily disposed on the optical element 10.

### Third Embodiment

An optical transmission module 1B according to a third embodiment and an endoscope 2B including the optical transmission module 1B will be described. The optical transmission module 1B and the endoscope 2B are similar to the optical transmission module 1 and the endoscope 2, respectively, and have the same functions as those of the optical transmission module 1 and the endoscope 2. Accordingly, the same components are denoted by the same reference numerals, and descriptions thereof are omitted.

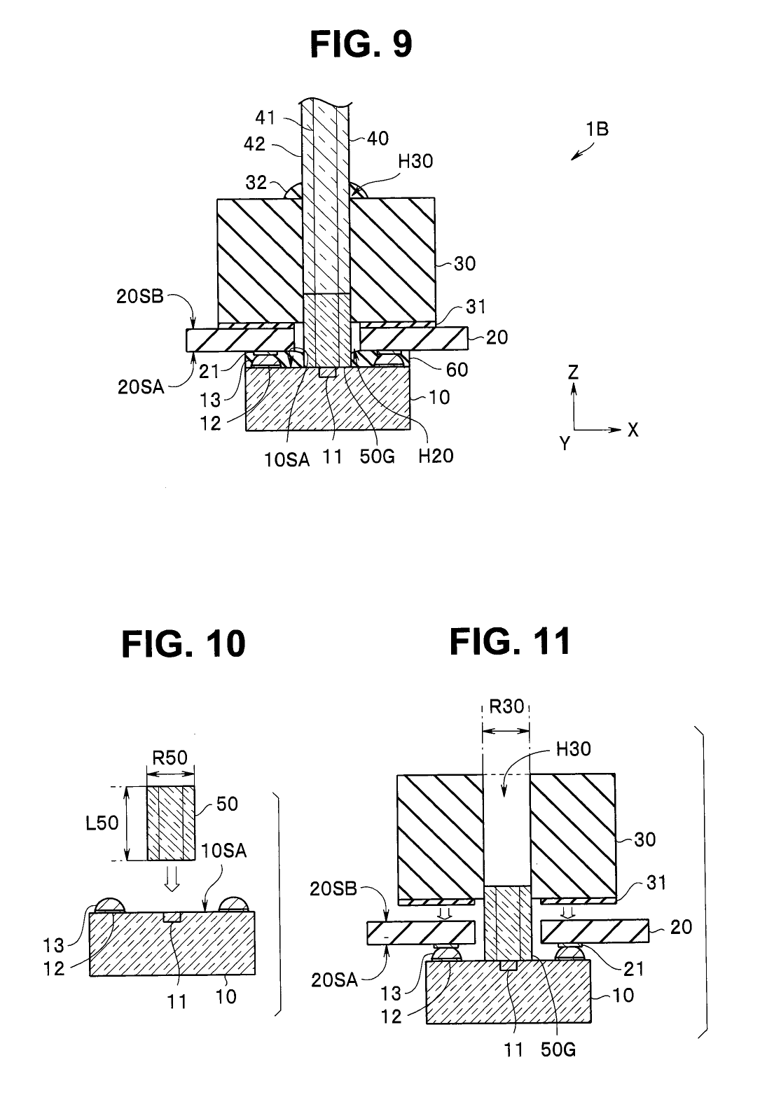

As illustrated in FIG. 9, in the optical transmission module 1B, an intermediate member 50G is long and an upper portion of the intermediate member 50G is inserted through the hole portion H20 of the wiring board 20 and inserted into the through-hole H30 of the holding member 30.

As illustrated in FIG. 10, the intermediate member 50G has the same configuration as that of the optical fiber 40, like the holding member 50C (see FIG. 5C). Specifically, the intermediate member 50G is composed of the core portion 41 and the clad portion 42 of the outer diameter R50. However, the length L50 of the intermediate member 50G is longer than the holding member 50C.

In a production method for the optical transmission module 1B, as illustrated in FIG. 10, first, the intermediate member 50G is joined to the light emitting surface 10SA in a state where the intermediate member 50G is positioned on the light emitting portion 11 of the optical element 10. Next, as illustrated in FIG. 11, an upper portion of the intermediate member 50G is inserted into the hole portion H20 of the substrate 20. After that, an upper portion of the intermediate member 50G is inserted into the through-hole H30 of the holding member 30. That is, like the outer diameter R40 of the optical fiber 40, the outer diameter R50 of the intermediate member 50G, has substantially “the same” size as the diameter of the through-hole H30 of the holding member 30. The intermediate member 50G is inserted and fitted into the through-hole H30, thereby allowing the optical element 10 and the holding member 30 to be automatically positioned in the horizontal direction (XY direction). Then, the wiring board 20 and the optical element 10 are bonded together.

The optical fiber 40 is inserted into the through-hole H30 and the distal end face thereof is brought into contact with the upper surface of the intermediate member 50G, thereby allowing the optical fiber 40 to be automatically positioned in the horizontal direction (XY direction), as well as in the vertical direction (Z-direction), with respect to the light emitting portion 11.

The optical transmission module 1B can be produced more easily than the optical transmission module 1 and the like.

Note that the same advantageous effects as those of the optical transmission module 1B can be obtained even by using an intermediate member which has the same configuration as that of the intermediate members 50 to 50D and is longer than the intermediate members 50 to 50D, instead of the intermediate member 50G. Specifically, in the optical transmission module in which the upper portion of the intermediate member is inserted through the hole portion of the wiring board and is inserted into the through-hole of the holding member, the optical fiber is inserted into the through-hole and the distal end face is brought into contact with the upper surface of the intermediate member, thereby allowing the optical fiber to be automatically positioned in the horizontal direction (XY direction), as well as in the vertical direction (Z-direction), with respect to the light emitting portion.

### Fourth Embodiment

An optical transmission module 1C according to a fourth embodiment and an endoscope 2C including the optical transmission module 1C will be described. The optical transmission module 1C and the endoscope 2C are similar to the optical transmission module 1B and the endoscope 2B, respectively, and have the same functions as those of the optical transmission module 1B and the endoscope 2B. Accordingly, the same components are denoted by the same reference numerals, and descriptions thereof are omitted.

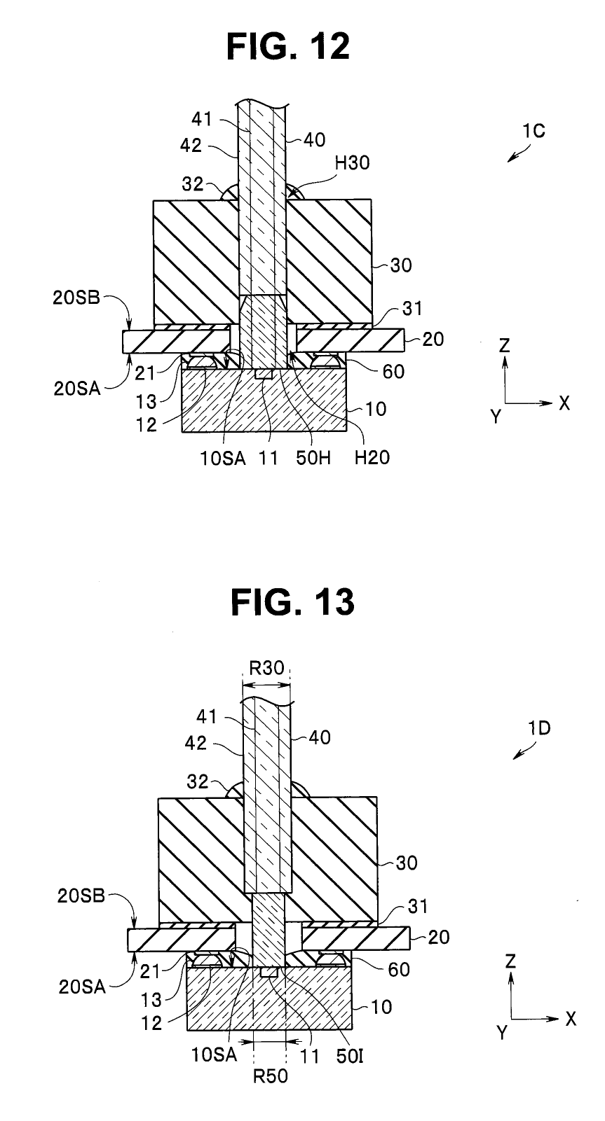

As illustrated in FIG. 12, in the optical transmission module 1C, the perimeter of an upper portion of an intermediate member 50H is tapered and the upper portion thereof is inserted through the hole portion H20 of the wiring board 20 and inserted and fitted into the through-hole H30 of the holding member 30.

In the optical transmission module 1C, the upper portion of the intermediate member 50H can be easily inserted into the through-hole H30 of the holding member 30. Accordingly, the optical transmission module 1C can be produced more easily than the optical transmission module 1B.

### Fifth Embodiment

An optical transmission module 1D according to a fifth embodiment and an endoscope 2D including the optical transmission module 1D will be described. The optical transmission module 1D and the endoscope 2D are similar to the optical transmission module 1B and the endoscope 2B, respectively, and have the same functions as those of the optical transmission module 1B and the endoscope 2B. Accordingly, the same components are denoted by the same reference numerals, and descriptions thereof are omitted.

As illustrated in FIG. 13, in the optical transmission module 1D, the outer diameter R50 of an intermediate member 501 is smaller than the outer diameter R40 of the optical fiber 40. The diameter of the through-hole H30 at the upper portion of the holding member 30 is different from that at the lower portion of the holding member 30. In other words, the diameter R50 at the upper portion of the through-hole H30 is substantially the same as the outer diameter R40 of the optical fiber 40, and a diameter R50A at the lower portion of the through-hole H30 is substantially the same as the outer diameter R50 of the intermediate member 501.

The optical transmission module 1D has the same advantageous effects as those of the optical transmission module 1B.

Note that, when the intermediate member 50A has a rectangular parallelepiped shape (FIG. 5B) and the intermediate member 50B has a polygonal columnar shape (FIG. 5C), the lower portion of the through-hole H30 of the holding member 30 is formed to have a rectangular or polygonal sectional shape, thereby obtaining the same advantageous effects as those of the optical transmission module 1B.

Note that an optical transmission module and the like including a light emitting element as the optical element 10 have been described above by way of example. However, the same advantageous effects can also be obtained even when the optical element is an O/E optical transmission module of a light receiving element including a light receiving portion such as a photo diode, as long as the optical element has a configuration similar to that described above.

The O/E optical transmission module disposed at the distal end portion of the endoscope transmits, for example, a clock signal to be input to an image pickup device as an optical signal. The endoscope that transmits the clock signal through the thin optical fiber 40 includes the thin insertion portion 80 and has minimal invasiveness.

As described above, the optical transmission module according to another embodiment of the present invention includes: a light receiving element having a front surface on which an optical fiber through which an optical signal is transmitted, a light receiving portion on which the optical signal is incident, and an external electrode are disposed; a holding member having a through-hole into which the optical fiber is inserted; a wiring board including a hole portion functioning as an optical path for the optical signal, the holding member being joined to a first principal surface, the external electrode of the optical element and a bond electrode being disposed on a second principal surface and bonded together; and a sealing resin for sealing a bond portion between the external electrode and the bond electrode. The optical transmission module further includes an intermediate member having an upper surface to be brought into contact with a distal end face of the optical fiber, and a lower surface to be brought into contact with the light receiving portion of the optical element.

The present invention is not limited to the embodiments, the modifications, and the like described above. Various changes, combinations, and applications are possible without departing from the scope of the invention.

What is claimed is:

1. An endoscope comprising:
an insertion portion including an optical transmission module at a distal end portion in which an image pickup device is disposed; and
an operation portion extending toward a proximal end portion side of the insertion portion, wherein
the optical transmission module includes:
an optical fiber including a core portion through which an optical signal is transmitted, and a clad portion covering an outer circumferential surface of the core portion, the optical fiber being inserted through the insertion portion;
an optical element having a front surface on which an optical element portion and an external electrode are disposed, the optical signal being output from the optical element portion, or being incident on the optical element portion;
a holding member having a through-hole into which the optical fiber is inserted;
a wiring board including a hole portion functioning as an optical path for the optical signal, the holding member being joined to a first principal surface, the external electrode of the optical element and a bond electrode being disposed on a second principal surface and bonded together; and
a sealing resin for sealing a bond portion between the external electrode and the bond electrode, wherein
the endoscope further comprises an intermediate member having an upper surface to be brought into contact with a distal end face of the optical fiber, and a lower surface to be brought into contact with the optical element portion of the optical element, and having a same configuration as a configuration of the optical fiber, and
an upper portion of the intermediate member is inserted into the through-hole of the holding member, a perimeter of the upper portion of the intermediate member being tapered.

2. An optical transmission module comprising:
an optical fiber configured to transmit an optical signal;
an optical element having a front surface on which an optical element portion and an external electrode are disposed, the optical signal being output from the optical element portion, or being incident on the optical element portion;
a holding member having a through-hole into which the optical fiber is inserted;
a wiring board including a hole portion functioning as an optical path for the optical signal, the holding member being joined to a first principal surface, the external electrode of the optical element and a bond electrode being disposed on a second principal surface and bonded together; and
a sealing resin for sealing a bond portion between the external electrode and the bond electrode,
wherein the optical transmission module further comprises an intermediate member having an upper surface to be brought into contact with a distal end face of the optical fiber, and a lower surface to be brought into contact with the optical element portion of the optical element.

3. The optical transmission module according to claim 2, wherein the intermediate member is made of a transparent material.

4. The optical transmission module according to claim 2, wherein the intermediate member is made of a transparent material having a same configuration as a configuration of the optical fiber.

5. The optical transmission module according to claim 2, wherein the intermediate member has a space functioning as the optical path.

6. The optical transmission module according to claim 2, wherein an upper portion of the intermediate member is fitted into the through-hole of the holding member.

7. The optical transmission module according to claim 2, wherein
the intermediate member has a same configuration as a configuration of the optical fiber,
the perimeter of the upper portion of the intermediate member is tapered, and
a part of the upper portion of the intermediate member is fitted into the through-hole of the holding member.
